(19) United States
(12) Patent Application Publication
(10) Pub. No.: US 2018/0236301 A1
(43) Pub. Date: Aug. 23, 2018

(54) BREATHING TRAINING DEVICE ADAPTERS AND ASSOCIATED METHODS

(71) Applicant: Duke University, Durham, NC (US)

(72) Inventors: Harrison Jones, Durham, NC (US); Matt Brown, Durham, NC (US)

(21) Appl. No.: 15/900,036

(22) Filed: Feb. 20, 2018

Related U.S. Application Data
(60) Provisional application No. 62/460,940, filed on Feb. 20, 2017.

(51) Int. Cl. A63B 23/18; A61M 16/20

(52) U.S. Cl. CPC A63B 23/18; A61M 16/208

(57) ABSTRACT

Breathing training device adapters and associated methods are disclosed herein. According to an aspect, a breathing training device adapter includes a housing comprising a first end and a second end that each define an opening. The housing defines an interior space that extends between the first end and the second end to enable gas flow between the opening of the first end and the opening of the second end. The adapter may include a first connector configured to operatively connect the opening of the first end to a breathing training mouthpiece as well as a gas valve operatively connected to the housing to provide controlled gas flow between the interior space of the housing and outside the housing. Further, the adapter may include a second connector configured to operating connect the opening of the second end to a breathing training device.

### CROSS REFERENCE TO RELATED APPLICATION

This application claims priority to U.S. Provisional Patent Application No. 62/460,940, filed Feb. 20, 2017, and titled DEVICE FOR RESPIRATORY MUSCLE TRAINING AND METHODS OF MAKING AND USING SAME, the disclosure of which is incorporated herein by reference in its entirety.

### STATEMENT AS TO FEDERALLY SPONSORED RESEARCH

This invention was made with the support of the United States government under Federal Grant No. 1R21AR069880-01 and awarded by the National Institutes of Health (NIH). The Government has certain rights in this invention.

### TECHNICAL FIELD

The presently disclosed subject matter relates to muscle training. More particularly, the presently disclosed subject matter relates to breathing training device adapters and associated methods.

### BACKGROUND

Respiratory muscle training (RMT) is performed when an exercise stimulus is delivered to the muscle of respiration and/or the upper airway to increase their strength, endurance, and/or performance. RMT regimens typically utilize handheld devices that provide resistance against inspiration and/or expiration. This is generally accomplished with the use of flow-resistive or pressure-threshold RMT devices. While the resistance provided by flow-resistive RMT devices is largely dependent on flow rate, pressure-threshold resistance is a relatively simple, low-cost approach to providing calibrated resistance relatively independent of flow rate. Pressure-threshold RMT repetitions occur when sufficient inspiratory or expiratory pressure is generated to overcome the resistance provided via a spring-loaded flange at a particular target (e.g., 30 cm H₂O). RMT regimens can be individualized and adjusted over time. Inspiratory muscle training (IMT) targets the muscles of inspiration (i.e., diaphragm) via generation of negative gas pressure resistance, while expiratory muscle training (EMT) targets the muscles of expiration (i.e., abdominal wall) via generation of positive gas pressure resistance. RMT, IMT, and EMT may be generally referred to as breathing training.

Current RMT device technology has limitations. For example, RMT devices may be designed to provide resistance against for inspiratory or expiratory repetitions only. Other RMT devices provide resistance during both inspiration and expiration, making repetitions too effortful for successful training. Our RMT device adapter addresses both of these limitations by 1) allowing an inspiratory type of device to be used for expiratory muscle training and vice versa, and 2) incorporation of one-way valve configurations to allow for minimal inspiratory resistance during expiratory repetitions, and vice versa. Thus, this technology expands the training capabilities of currently available RMT devices.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Disclosed herein are breathing training device adapter and associated methods. According to an aspect, a breathing device adapter includes a housing comprising a first end and a second end that each define an opening. Such an adapter may be connected to a RMT device, a IMT device, or an EMT device. The housing defines an interior space that extends between the first end and the second end to enable gas flow between the opening of the first end and the opening of the second end. The adapter may include a first connector configured to operatively connect the opening of the first end to a breathing training mouthpiece. Further, the adapter may include a second connector configured to operating connect the opening of the second end to a breathing training device.

### BRIEF DESCRIPTION OF THE DRAWINGS

The foregoing summary, as well as the following detailed description of various embodiments, is better understood when read in conjunction with the appended drawings. For the purposes of illustration, there is shown in the drawings exemplary embodiments; however, the presently disclosed subject matter is not limited to the specific methods and instrumentalities disclosed. A brief description of the drawings follows.

FIG. 1A is a top perspective view of a breathing training device adapter in accordance with embodiments of the present disclosure;

FIG. 1B is a bottom perspective view of the device shown in FIG. 1A;

FIG. 1C is a cross-sectional side view of the adapter shown in FIGS. 1A and 1B;

FIG. 1D is a perspective, cross-sectional side view of the adapter shown in FIGS. 1A, 1B, and 1C;

FIG. 2 is a perspective view of an end of an adapter having a gasket fitted thereto in accordance with embodiments of the present disclosure;

FIG. 3 is a perspective view of an end of an adapter having another gasket fitted thereto in accordance with embodiments of the present disclosure;

FIG. 4 is a side view of an adapter operatively connected to a mouthpiece at one end and an RMT device at another end; and

FIG. 5 is a side view of an adapter operatively connected to a mouthpiece at one end and another type of RMT device at another end.

### DETAILED DESCRIPTION

The presently disclosed subject matter is described with specificity to meet statutory requirements. However, the description itself is not intended to limit the scope of this patent. Rather, the inventors have contemplated that the claimed subject matter might also be embodied in other ways, to include different steps or elements similar to the ones described in this document, in conjunction with other present or future technologies.

Articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

As used herein, the term “user,” “subject,” and “patient” are used interchangeably herein and refer to an individual (e.g., human) in need of, or undergoing, respiratory muscle therapy by RMT, IMT, or EMT.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any nonclaimed element as essential to the practice of the presently disclosed subject matter.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

Unless otherwise defined, all technical terms used herein have the same meaning as commonly understood by one of ordinary skill in the art to which this disclosure belongs.

The presently disclosed subject matter provides RMT, IMT, and EMT device adapters and methods of use. These adapters may be used together with a RMT device to allow for the delivery of inspiratory and expiratory resistance across the range of human performance. These adapters may also be used to provide connection to a mouthpiece for RMT. An adapter in accordance with embodiments of the present disclosure may include a gas valve for providing controlled gas flow between an interior space of the adapter and outside the adapter. Particularly, the gas valve can provide one-way gas flow such that advantageously there is minimal inspiratory resistance when completing expiratory training and minimal expiratory resistance when performing inspiratory training.

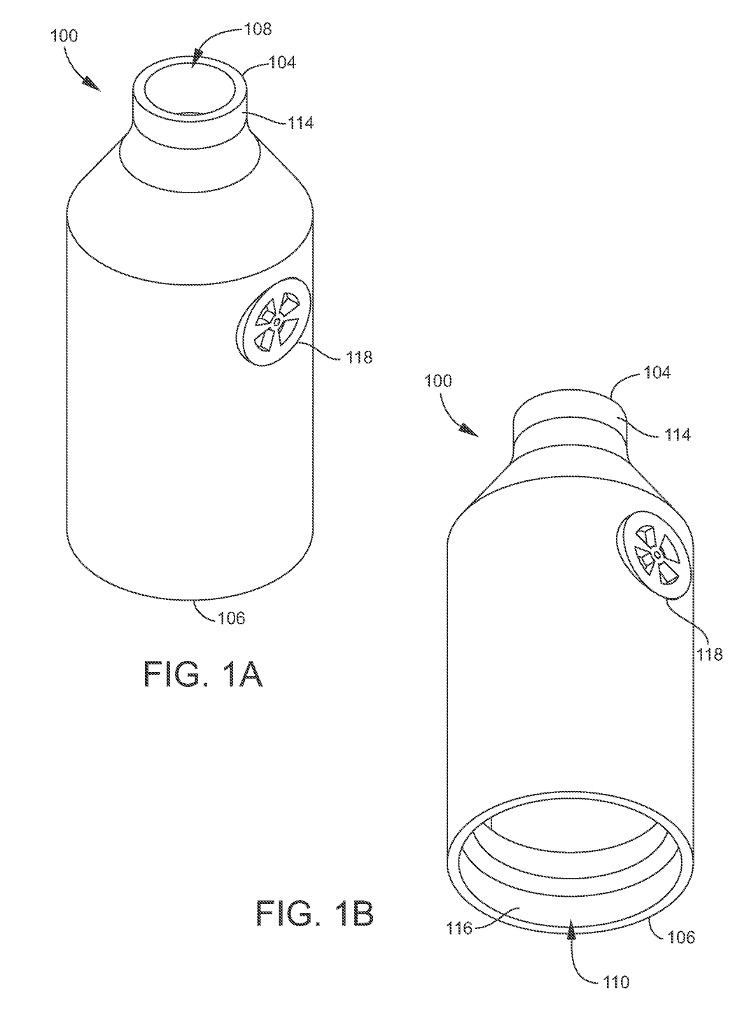

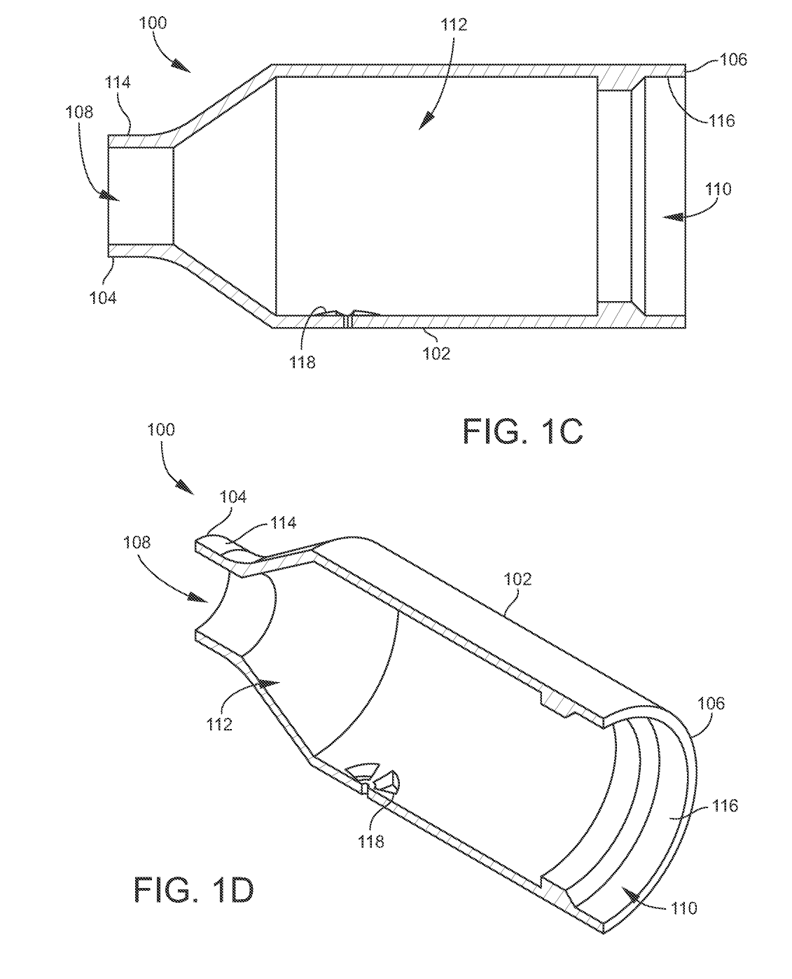

In accordance with embodiments of the present disclosure, FIGS. 1A-1D illustrate a breathing training device adapter 100 in accordance with embodiments of the present disclosure. Referring initially to FIGS. 1A and 1B, the figures show a top perspective view and a bottom perspective view of the adapter 100, respectively. The adapter includes a housing 102 having an end 104 and an opposing end 106. The housing 102 is substantially cylindrical in shape and tapers at the end 104 as shown in the figures, but it should be appreciated that the housing 102 may alternatively be any other suitable shape and size for holding by a person.

In this example, the entirety of the housing 102 and its ends 104 and 106 is made of one piece of plastic, but it should be appreciated that the housing 102 and its ends 104 and 106 may be made of multiple pieces of plastic and/or metals that are suitably arranged and attached for achieving functionality described herein and to contain and maintain a desired pressure. Example materials for the housing 102 include, but are not limited to, natural polymers such as shellac, amber, wool, silk, natural rubber, cellulose, synthetic polymers such as synthetic rubber, phenol formaldehyde resin (or Bakelite), neoprene, nylon, polyvinyl chloride (PVC or vinyl), polystyrene, polyethylene, polypropylene, polyacrylonitrile, PVB, silicone, (Polytetrafluoroethylene (Teflon), Polyethylene), vinyl, acrylic, polyester (polylactic acid or polylactide (PLA)), and the like. During operation, the pressure inside the housing may be in the range of between about 10 cm H₂O and about 300 cm H₂O. For example, the pressure may be about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300 cm H₂O. In some embodiments, the pressure within the device is between about 30-150 cm H₂O.

FIGS. 1C and 1D illustrate a cross-sectional side view and a perspective, cross-sectional side view, respectively, of the adapter 100. Now referring to FIGS. 1C and 1D, ends 104 and 106 define an openings 108 and 110, respectively. The openings 108 and 110 are circular in shape in this example, but it should be appreciated that the openings 108 and 110 may alternatively be any other suitable shape for fitting to another desired RMT component. The housing 102 defines an interior space 112 that extends between the ends 104 and 106 to enable gas flow between the opening of end 104 and end 106. As a result, in the example of RMT, gas can freely pass between a person's mouth or mouthpiece and an RMT device. As shown in FIGS. 1C and 1D, the interior space 112 encompasses nearly the entirety of the inside of the housing 102; however, the open area within the interior space 112 may be smaller, or of any other suitable shape and size.

The end 104 may be sized and shaped to be a connector for operatively connecting the opening 108 to an RMT mouthpiece (not shown). In this example, an outer surface 114 of the end 104 is sized and shaped to engage securely to the RMT mouthpiece. In particular, a mating surface of the RMT mouthpiece can tightly fit around the outer surface 114 such that an airtight seal can form between the RMT mouthpiece and the opening 108 to thereby allow gas to operatively pass between the interior space 112 and the interior of the mouthpiece.

The end 106 may be sized and shaped to be a connector for operatively connecting the opening 110 to a RMT device (not shown). In this example, an interior surface 116 of the end is size and shaped to securely hold a gasket (not shown) for engagement with the RMT device. The gasket can tightly fit around an outer surface of the RMT device such that a gas seal can form between the RMT device and the opening 110 to thereby allow gas to operatively pass between the interior space 112 and the interior of the RMT device. The gasket can be made of rubber or any other suitable material, such as a soft plastic. As a result of attaching the mouthpiece and the RMT device to the connectors as described, gas may flow between the mouthpiece and the RMT device via by passage through the interior space 112.

The adapter 100 may include a gas valve 118 operatively connected to the housing 102 to provide controlled gas flow between the interior space 112 and outside the housing 102. In this example, the gas valve 118 is a one-way gas valve configured to permit gas flow from the interior space 112 to outside the housing 102 during exhale operation of breathing training. Also, in this example, the gas valve 118 is configured to restrict gas flow from outside the housing to the interior space 112 during inhale operation of breathing training.

In accordance with embodiments, an adapter as disclosed herein may be configured to operate with a data collection/dose control/feedback tool that can monitor desired data points, such as the number of respirations taken, the number of respirations remaining in the exercise program, the force of each inspiration/expiration, and the like.

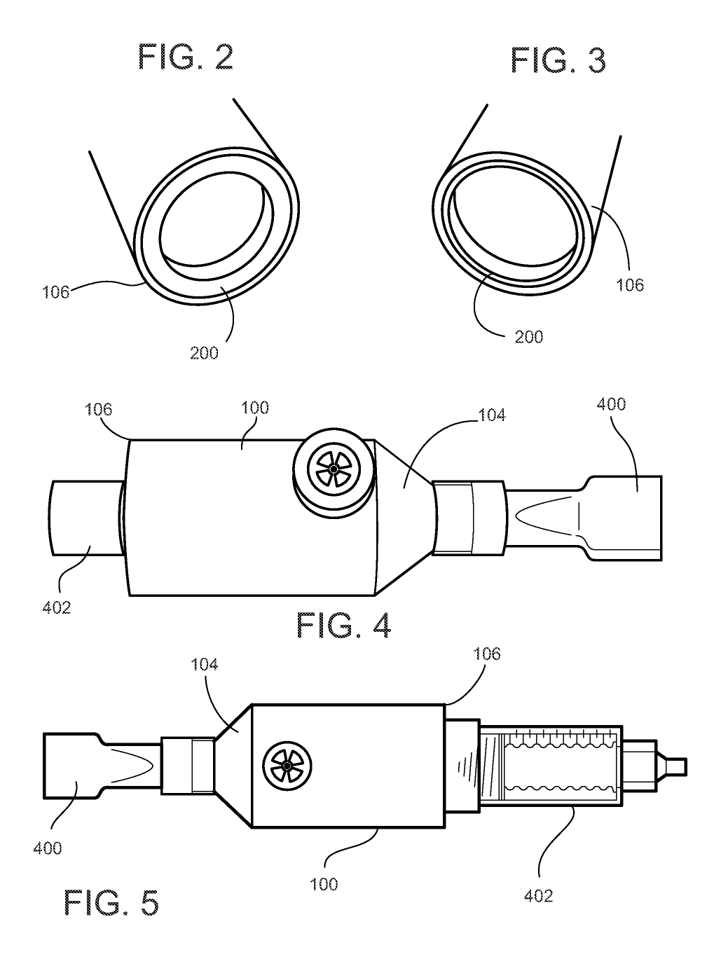

FIGS. 2 and 3 each illustrate a perspective view of an end 106 of an adapter 100 in accordance with embodiments of the present disclosure. Referring to FIGS. 2 and 3, a gasket 200 is fitted in the end 106 as described herein for fitting tightly around an outer surface of an RMT device. As shown, the different gaskets 200 have different sizes for fitting different RMT devices.

FIG. 4 illustrates a side view of an adapter 100 operatively connected to a mouthpiece 400 at end 104 and an RMT device 402 at end 106. In this example, the RMT device 402 is an EMST150™ device.

FIG. 5 illustrates a side view of an adapter 100 operatively connected to a mouthpiece 400 at end 104 and another RMT device 402 at end 106. In this example, the RMT device 402 is a Threshold PEP device.

In accordance with embodiments, an adapter as disclosed herein may be used with a mouthpiece and an RMT device in a RMT therapy session. For example, the systems shown in FIGS. 4 and 5 may be used in a RMT therapy session. Generally, referring to FIGS. 1A-1D, such a method includes inserting a RMT device (e.g., device 402) into the second opening 110 of the adapter 100. The method also includes inserting a mouthpiece (e.g., mouthpiece 400) over the outer surface 114 of the end 104. Further, the method includes placing the mouthpiece into the mouth of a subject. The subject may subsequently inhale and exhale for a predetermined amount of breaths into the mouthpiece. In some embodiments, data is recorded and analyzed based on the RMT exercise.

In example uses of a system having an adapter disclosed herein, a RMT therapy sessions may be conducted by the RMT clinician and each visit may take approximately 45 minutes. Therapy visits can include the following sequential steps:
Measurement of MIP and MEP by the RMT clinician. MIP and MEP measurement will occur at the beginning of each RMT therapy session in addition to the 4 assessments previously described. This allows the RMT clinician to provide progressive resistance for subjects in both study arms.
Download and review of RMT adherence and performance data from data collection/dose control/feedback tool via USB.
Calibration of RMT pressure-threshold device and programming of data collection/dose control/feedback tool.
Completion of 1 set of 25 repetitions for each inspiratory and expiratory RMT/sham-RMT; behavioral observations will be made by clinician of RMT/sham-RMT tolerance throughout.
Subjects may be asked to rate pain and perceived effort associated with RMT using a standard 0-10 scale, intermittently and after each RMT/sham-RMT set.
Based on results of steps 4 and 5, the RMT/sham-RMT training stimulus will be modified (details below) if pain rating is ≥4, perceived effort rating is ≥8, and/or behavioral observations suggest excessive effort. Additionally, in the control arm only, the sham-RMT training stimulus may be modified if subjects exhibit minimal effort suggestive of attempts by the subject to penetrate the blind.
Completion of alternating sets of 25 repetitions of inspiratory and expiratory RMT/sham-RMT while steps 5 & 6 are repeated until 3 well-tolerated sets of 25 successful inspiratory and expiratory RMT/sham-RMT repetitions are achieved.

While the embodiments have been described in connection with the various embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed:

1. A breathing device adapter comprising:
a housing comprising a first end and a second end that each define an opening, the housing defining an interior space that extends between the first end and the second end to enable gas flow between the opening of the first end and the opening of the second end;
a first connector configured to operatively connect the opening of the first end to a breathing training mouthpiece; and
a second connector configured to operating connect the opening of the second end to a breathing training device.

2. The adapter of claim 1, wherein the housing is made of at least one of plastic and metal.

3. The adapter of claim 1, further comprising a gasket fitted to the second end for fluidly sealing the breathing training device to the second opening when the breathing training device is operatively connected to the second connector.

4. The adapter of claim 3, wherein the gasket is made of rubber.

5. The adapter of claim 1, further comprising a gas valve operatively connected to the housing to provide controlled gas flow between the interior space of the housing and outside the housing.

6. The adapter of claim 5, wherein the gas valve is a one-way valve configured to permit gas flow from the interior space of the housing to outside the housing during exhale operation of breathing training, and wherein the one-way gas valve is configured to restrict gas flow from outside the housing to the interior space of the housing during inhale operation of breathing training.

7. A breathing training system comprising:
a breathing training mouthpiece;
a breathing training device; and
a breathing training device adapter comprising:
a housing comprising a first end and a second end that each define an opening, the housing defining an interior space that extends between the first end and the second end to enable gas flow between the opening of the first end and the opening of the second end;
a first connector configured to operatively connect the opening of the first end to the breathing training mouthpiece; and
a second connector configured to operating connect the opening of the second end to the breathing training device.

8. The system of claim 7, further comprising a gasket fitted to the second end for fluidly sealing the breathing training device to the second opening when the breathing training device is operatively connected to the second connector.

9. The system of claim 8, wherein the gasket is made of rubber.

10. The system of claim 7, further comprising a gas valve operatively connected to the housing to provide controlled gas flow between the interior space of the housing and outside the housing.

11. The system of claim 10, wherein the gas valve is a one-way valve configured to permit gas flow from the interior space of the housing to outside the housing during exhale operation of breathing training, and wherein the one-way valve is configured to restrict gas flow from outside the housing to the interior space of the housing during inhale operation of breathing training.

12. A method of breathing training, the method comprising:
providing a breathing training device adapter comprising:
a housing comprising a first end and a second end that each define an opening, the housing defining an interior space that extends between the first end and the second end to enable gas flow between the opening of the first end and the opening of the second end;
a first connector configured to operatively connect the opening of the first end to a breathing training mouthpiece; and
a second connector configured to operating connect the opening of the second end to a breathing training device;
connecting the first connector to the breathing training mouthpiece;
connecting the second connector to the breathing training device; and
receiving from a user one of inhales out of or exhales into the breathing training mouthpiece.

13. The method of claim 12, wherein the housing is made of at least one of plastic and metal.

14. The method of claim 12, further comprising a gasket fitted to the second end for fluidly sealing the breathing training device to the second opening when the breathing training device is operatively connected to the second connector.

15. The method of claim 14, wherein the gasket is made of rubber.

16. The method of claim 12, further comprising a gas valve operatively connected to the housing to provide controlled gas flow between the interior space of the housing and outside the housing.

17. The method of claim 16, wherein the gas valve is a one-way gas valve configured to permit gas flow from the interior space of the housing to outside the housing during exhale operation of breathing training, and wherein the one-way valve is configured to restrict gas flow from outside the housing to the interior space of the housing during inhale operation of breathing training.
